(19) United States
(12) Patent Application Publication
(10) Pub. No.: US 2026/0183337 A1
(43) Pub. Date: Jul. 2, 2026

(54) TUMOR MEMORY KILLER CELL AND METHOD OF PRODUCING THE SAME

(71) Applicant: DONG-A UNIVERSITY RESEARCH FOUNDATION FOR INDUSTRY-ACADEMY COOPERATION, Busan (KR)

(72) Inventors: Seok Ho KIM, Busan (KR); Ju Heon LEE, Busan (KR); Da Na JUNG, Busan (KR); Sung Yong OH, Busan (KR)

(73) Assignee: DONG-A UNIVERSITY RESEARCH FOUNDATION FOR INDUSTRY-ACADEMY COOPERATION, Busan (KR)

(21) Appl. No.: 19/006,971

(22) Filed: Dec. 31, 2024

(51) Int. Cl. A61K 35/17; A61K 40/11; A61K 40/31; A61K 40/42; A61P 35/00; C07K 14/705; C07K 14/725; C12N 5/00; C12N 5/0783; C12N 13/00

(52) U.S. Cl. CPC A61K 35/17; A61K 40/11; A61K 40/31; A61K 40/421; A61K 40/4224; A61P 35/00; C07K 14/7051; C07K 14/70589; C12N 5/0018; C12N 5/0636; C12N 13/00; C12N 2501/2302; C12N 2501/2315; C12N 2501/2318; C12N 2501/2321; C12N 2502/30; C12N 2506/1369

(57) ABSTRACT

Provided are a cord blood-derived tumor memory killer cell and a method of producing the same. According to a tumor memory killer cell according to an aspect and a method of producing the same, tumor memory killer cells with improved memory ability for tumor cells is provided, and thus can be effectively used as an anticancer immune cell therapeutic agent which induces cancer cell death through the same mechanism as existing cytokine-induced killer cells, but exhibits a stronger effect of inducing tumor cell death.

### BACKGROUND

### 1. Field

The present disclosure relates to a tumor memory killer cell and a method of producing the same.

### 2. Description of the Related Art

Immune cell therapy has recently emerged as one of the important methods of treating cancer in addition to existing standard therapies. Immune cell therapy is based on the body's own response to cancer tissues, and thus has the advantage of avoiding serious side effects caused by other treatment methods. Immune cells used in anticancer treatment include lymphokine-activated killer (LAK) cells, tumor-infiltrating lymphocytes, antigen-specific toxic T lymphocytes (CTLs), cytokine-induced killer (CIK) cells, and the like, and the efficacy and side effects thereof vary depending on the type and condition of cancer.

Cytokine-induced killer (CIK) cells are T cells that have the characteristics and functions of natural killer cells and are known to have the ability to effectively destroy cancer cells and be capable of eliminating various types of cancer cells regardless of MHC. It is also known that clinical trials are underway or completed in Korea and abroad for lymphoma, pancreatic cancer, hepatocellular cancer, myeloma, renal cell cancer, non-small cell lung cancer, gastric cancer, progressive pancreatic cancer, and the like.

However, studies have shown that previously reported cytokine-induced killer cells have poor cytotoxicity against cells derived from actual cancer patients. Therefore, the inventors of the present disclosure confirmed that tumor memory killer cells having memory ability for specific cancer patient cells, obtained by culturing cord blood mononuclear cells together with feeder cells and cytokines, exhibited an excellent anticancer effect, thus completing the present disclosure.

### SUMMARY

An aspect is to provide an isolated tumor memory killer cell that has a surface antigen characteristic of CD56+CD3+, CD45RA−, or CD45RO+.

Another aspect is to provide a method of producing a tumor memory killer cell, the method including (a) obtaining a cord blood mononuclear cell (CBMC) and (b) co-culturing the obtained CBMC and a feeder cell in a medium containing a cytokine.

Another aspect is to provide a composition for producing a tumor memory killer cell, the composition including IL-2, IL-15, and a feeder cell obtained by irradiating a solid cancer cell or a hematologic cancer cell with radiation.

Another aspect is to provide a composition for treating cancer, including, as an active ingredient, a tumor memory killer cell produced by the method of producing a tumor memory killer cell, or a population thereof.

Additional aspects will be set forth in part in the description which follows and, in part, will be apparent from the description, or may be learned by practice of the presented embodiments of the disclosure.

An aspect provides a method of producing tumor memory killer cells using cord blood mononuclear cells isolated from cord blood together with cytokines and feeder cells.

An aspect provides an isolated tumor memory killer cell that has a surface antigen characteristic of CD56+CD3+, CD45RA−, or CD45RO+.

In an embodiment, the isolated tumor memory killer cell may be positive for surface marker Nkp36, Nkp40 or Nkp46.

The term “positive or +” as used herein may mean that, with regard to a cell marker, the cell marker is present in a large amount or at a high concentration, as compared to that in other cells as a reference. In other words, any marker is present inside or on the surface of a cell, and therefore, when a cell may be distinguished from one or more other cell types by using the marker, the cell may be positive for the marker. The term “positive” may also mean that a cell has the marker in an amount sufficient to produce a signal, e.g., a signal from a cytometer, at a value greater than the background value. For example, cells may be detectably labeled with CD45RO-specific antibodies, and when signals from these antibodies are detectably stronger than those of a control (e.g., background value), the cells may be “CD45RO+.” The term “negative or −” as used herein may mean that, although antibodies specific to a particular cell surface marker are used, the marker cannot be detected, as compared to the background value. For example, when a cell cannot be detectably labeled with a CD45RA-specific antibody, the cells may be “CD45RA−.”

In an embodiment, the tumor memory killer cell may be an activated tumor memory killer cell. The activated tumor memory killer cell may refer to a cell in which cytotoxicity, intrinsic immunomodulatory ability of killer cells, or tumor memory is activated, as compared to parent cells, for example, cord blood cells, or lymphocytes before activation. In a specific embodiment, the activated tumor memory cells are CD56+CD3+. In a specific embodiment, the activated tumor memory killer cells are CD56+CD3+CD45RO+. In other specific embodiments, the activated natural killer cells may further include Nkp30+Nkp44+Nkp46+. In specific embodiments, greater than 50%, 60%, 70%, 80%, 90%, 92%, 94%, 96% or 98% of the activated tumor memory killer cells are CD56+ and CD3+. In other embodiments, at least 50%, 60%, 70%, 80%, 82%, 84%, 86%, 88% or 90% of the activated tumor memory killer cells are CD45RO+ and CD45RA−. In other embodiments, at least 50%, 52%, 54%, 56%, 58% or 60% of the activated tumor memory killer cells are Nkp30+. In other embodiments, at least 50%, 52%, 54%, 56%, 58% or 60% of the activated tumor memory killer cells are Nkp44+. In other embodiments, at least 50%, 52%, 54%, 56%, 58% or 60% of the activated tumor memory killer cells are Nkp46+.

In an embodiment, activated tumor memory killer cells or a population enriched with activated tumor memory killer cells may be evaluated by detecting one or more functionally related markers, for example, CD56, CD3, CD45RA, CD45RO, Nkp30, Nkp44, and Nkp46.

In an embodiment, the isolated tumor memory killer cell may be derived from any one selected from the group consisting of umbilical cord, cord blood, placenta, amniotic fluid, and amniotic membrane. Specifically, the isolated tumor memory killer cell may be derived from cord blood.

In an embodiment, an occupancy rate of memory T cells in a population of the isolated tumor memory killer cell may be 50% or more.

In an embodiment, an occupancy rate of central memory T cells in the memory T cells may be 50% or more.

In an embodiment, the tumor memory killer cell may be genetically modified or engineered.

In this specification, natural killer cells may be genetically modified or engineered.

The term “genetic modification” or “genetic engineering” as used herein includes artificial alteration of the composition or structure of the genetic material of a cell.

In an embodiment, the tumor memory killer cell may be genetically modified to have improved targeting specificity and/or homing specificity.

In an embodiment, the isolated tumor memory killer cell or a population thereof may have increased activity compared to cytokine-induced killer cells, or may be genetically modified to have increased activity.

In an embodiment, the genetically modified tumor memory killer cell is a tumor memory killer cell including a chimeric antigen receptor (CAR). The CAR is an artificial membrane-binding protein that induces immune cells (e.g., T lymphocytes) against an antigen, and kills cells expressing the antigen by stimulating the immune cells. The CAR includes an extracellular domain that binds to an antigen, e.g., an antigen on a cell, a transmembrane domain, and an intracellular (cytoplasmic) signaling domain (i.e., an intracellular stimulatory domain) that transmits primary activation signals to immune cells, and/or a co-stimulatory domain. When all other conditions are satisfied, the CAR is expressed on the surface of, for example, T lymphocytes, such as primary T lymphocytes, and the extracellular domain of the CAR binds to an antigen, the extracellular signaling domain activates and/or proliferates T lymphocytes by transmitting signals thereto, and kills cells expressing the antigen when the antigen is present on the cell surface. Some immune cells, e.g., T lymphocytes and natural killer cells, require two signals, i.e., a primary activation signal and a co-stimulatory signal, to maximize activation, and CARS may also include a co-stimulatory domain such that binding of the antigen to the extracellular domain results in transmission of both the primary activation signal and the co-stimulatory signal.

In an embodiment, the genetically modified tumor memory killer cell may be a tumor memory killer cell including a homing receptor. This causes the cell including a homing receptor to home to a particular anatomical zone, particularly, a tissue, or a particular type of cell, e.g., B cell zone of lymph nodes, gastrointestinal tract, or skin.

In an embodiment, the genetically modified tumor memory killer cell is a tumor memory killer cell including both a CAR and a homing receptor as described herein.

The tumor memory killer cell including a CAR and/or a homing receptor may be produced by any method known in the art. In some embodiments, the tumor memory killer cell including a CAR and/or a homing receptor is engineered to express the CAR and/or the homing receptor by introducing (e.g., transfection), into the tumor memory killer cell, one or more vectors including a nucleic acid sequence encoding the CAR and/or the homing receptor. In some embodiments, cells, from which tumor memory killer cells may be produced, may be first engineered to express a CAR and/or a homing receptor by introducing (e.g., transfection), into the cells, one or more vectors including a nucleic acid sequence encoding the CAR and/or the homing receptor, and then tumor memory killer cells including the CAR and/or the homing receptor may be derived therefrom by the method as described above.

In an embodiment, the extracellular domain of the CAR is an antigen-binding domain. In a specific embodiment, the antigen-binding domain is an scFv domain. In specific embodiments, the antigen-binding domain specifically binds to a TAA. In specific embodiments, the TAA is selected from the group consisting of CD123, CLL-1, CD38, CD20, and CS-1. In a more specific embodiment, the antigen-binding domain includes a single-chain Fv (scFv) or antigen-binding fragment derived from an antibody that binds to CS-1. In a more specific embodiment, the antigen-binding domain includes a single-chain of elotuzumab and/or an antigen-binding fragment of elotuzumab. In specific embodiments, the antigen-binding domain includes a single-chain Fv (scFv) or antigen-binding fragment derived from an antibody that binds to CD20.

In an embodiment, the intracellular stimulatory domain of the CAR is a CD3 zeta signaling domain.

In an embodiment, the co-stimulatory domain of the CAR includes the intracellular domain of CD28, 4-1BB, PD-1, OX40, CTLA-4, NKp46, NKp44, NKp30, DAP10, or DAP12.

In an embodiment, the homing receptor is a chemotactic receptor. In specific embodiments, the chemotactic receptor is selected from the group consisting of CXCR4, VEGFR2, and CCR7.

In an embodiment, the genetically modified tumor memory killer cell or a population thereof may be engineered to express a CAR or a homing receptor.

An aspect provides a method of producing a tumor memory killer cell, the method including: (a) obtaining a cord blood mononuclear cell (CBMC); and (b) co-culturing the obtained CBMC and a feeder cell in a medium containing a cytokine.

The term “cord blood mononuclear cell (CBMC)” as used herein refers to a cell with a spherical nucleus existing in blood derived from cord blood, and these CBMCs include immune cells such as B cells, T cells, macrophages, dendritic cells, natural killer (NK) cells, and the like.

The term “feeder cells (also referred to as culture helper cells or supporting cells)” as used herein refers to cells that do not have the ability to divide and proliferate but are metabolically active, and thus produce various metabolites to help the proliferation of activated lymphocytes. Feeder cells that may be used in the present disclosure include animal cell lines into which genes are introduced, peripheral blood leukocytes (PBLs) treated with various cytokines or compounds, autologous or allogeneic peripheral blood solid cancer cells, hematologic cancer cells, leukocytes, T cells, B cells, monocytes, or the like, and preferably, may be autologous solid cancer cells or autologous hematologic cancer cells, but the present disclosure is not limited thereto. It is obvious that other feeder cells known to be commonly available in the art to which the present disclosure pertains may be used without limitation if the feeder cells are suitable for the objective of the present disclosure.

In an embodiment, the CBMC may be derived from cord blood.

In an embodiment, the feeder cell may be selected from the group consisting of colon cancer, ovarian cancer, liver cancer, biliary tract cancer, lung cancer, pancreatic cancer, or prostate cancer.

In an embodiment, the feeder cell may be collected from the ascites of patients with solid cancer. Specifically, the solid cancer may be selected from, but is not limited to, the group consisting of colon cancer, ovarian cancer, liver cancer, biliary tract cancer, lung cancer, pancreatic cancer, or prostate cancer. For example, the feeder cell may be selected from the ascites of patients with colon cancer, ovarian cancer, liver cancer, biliary tract cancer, lung cancer, pancreatic cancer, or prostate cancer.

The term “ascites” as used herein may refer to a condition in which body fluid accumulates abnormally in the abdominal cavity. Specifically, ascites often occurs due to causes such as liver lesions, heart failure, kidney failure, and cancer, and when ascites occurs, abdominal distension, discomfort, breathing difficulties, and the like may appear. For example, the ascites may be caused by colon cancer, ovarian cancer, liver cancer, biliary tract cancer, lung cancer, pancreatic cancer, or prostate cancer, but the present disclosure is not limited thereto.

In an embodiment, the feeder cell may be irradiated with radiation. Specifically, the radiation may be gamma rays, and a radiation dose may be preferably in a range of about 20 Gy to about 200 Gy, more preferably in a range of about 20 Gy to about 100 Gy, and most preferably 50 Gy, but the present disclosure is not limited thereto.

In an embodiment, the co-culture may be to produce a tumor memory killer cell by culturing a CBMC.

The co-culture of the CBMC may be performed under normal cell culture conditions, i.e., at about 37° C. in a CO₂ incubator, and may be continuously cultured while a culture medium is added once every 2 days or 3 days. In addition, the concentration of CBMCs added to the medium may be in a range of about 4×10⁵ cells/ml to about 5×10⁷ cells/ml, but the present disclosure is not limited thereto. The culture may be performed for, for example, about 3 days to about 28 days, about 5 days to about 25 days, about 7 days to about 23 days, about 10 days to about 21 days, or 21 days, but the present disclosure is not limited thereto. To obtain the desired number of tumor memory killer cells, the number of culture days may be appropriately set within or outside the above ranges. Culture vessels may include commercially available dishes, flasks, plates, multi-well plates, and culture bags.

In an embodiment, a ratio of the CBMCs to the feeder cells in the co-culture may be in a range of about 1:1 to about 8:1.

In an embodiment, a cytokine that may be included in the medium is preferably one or more selected from interleukins. Interleukin is a generic term for proteinaceous bioactive substances produced by immune cells such as lymphocytes, monocytes, and macrophages, and interleukin that may be used in the present disclosure may be one or more selected from the group consisting of IL-2, IL-15, IL-21, Flt3-L, IL-7, IL-12, and IL-18. In particular, IL-2 and IL-15 or IL-2, IL-15, IL-18, and IL-21 may be used, and IL-21 and IL-18 may not be continuously used but be used for a short period of time (discontinuation after added once to a culture medium on the start of the culture), but the present disclosure is not limited thereto. It is obvious that other cytokines may also be used by those of ordinary skill in the art to which the present disclosure pertains without limitation as long as the cytokines are suitable for the objective of the present disclosure.

It has been reported that IL-2 has the function of enhancing the proliferation and activation of mature NK cells. There are reports that the number of NK cells is significantly reduced in humans and mice deficient in IL-2. Meanwhile, studies proved that IL-2 and IL-2Ra deficiency indirectly affects the number and activation of NK cells, and IL-2R chain is known to be involved in the formation of IL-15 receptor.

With regard to IL-15, it was found that NK cells were not found in mice deficient in IL-15 or IL-15Rα, and NK cells were deficient in mice deficient in transcription factor interferon (IFN)-regulatory factor 1 that is required for IL-15 production. Accordingly, it is known that IL-15 is involved in NK cell differentiation and that IL-15 directly promotes the growth and differentiation of NK cells through the IL15 receptor expressed on NK cells.

In an embodiment of the present disclosure, CBMCs and feeder cells were co-cultured in the presence of cytokines IL-2 and IL-15, and through fluorescence-activated cell sorting (FACS) and mean fluorescence intensity (MFI), it was confirmed that the proportion of CD3-CD56+ cells increased compared to cytokine-induced killer cells in activated lymphocytes. Also, compared to cytokine-induced killer cells, the expression of CD45RA was relatively low and the expression of CD45RO increased, and thus as a result of examining the effector memory phenotype, the production of tumor memory killer cells was confirmed.

In an embodiment, the culture may be performed step by step to maximize the proliferation of the tumor memory killer cells. In this case, culture medium components, culture vessels, and culture periods in respective culture stages may be the same as or different from each other, and the optimal culture period for each stage may be found, thereby completing the proliferation of natural killer cells. In addition, in the present disclosure, the culture may be performed by adding a culture composition (or culture medium) into a flask at intervals of 2 days to 3 days, which serves to help natural killer cells to be effectively cultured.

In an embodiment, the culture may include an initial stage for about 2 days to about 7 days and a subsequent stage for about 10 days to about 20 days.

In an embodiment, the initial stage and subsequent stage of the culture may differ in the composition of the cytokine contained in the medium.

In an embodiment, the initial stage of the culture may include two or more cytokines selected from the group consisting of IL-2, IL-21, and IL-18. Specifically, the initial stage of the culture may include IL-2, IL-21, and IL-18 in the medium.

In an embodiment, the initial stage of the culture may include IL-2 and IL-15 in the medium.

In an embodiment, the initial stage and subsequent stage of the culture may differ in a ratio of CBMCs to feeder cells contained in the medium.

In an embodiment, the ratio of CBMCs to feeder cells in the initial stage of the culture may be in a range of about 5:1 to about 8:1. Specifically, the ratio of CBMCs to feeder cells may be 6:1. Specifically, the number of CBMCs may be 3×10⁶, and the number of feeder cells may be 5×10⁵.

In an embodiment, the ratio of CBMCs to feeder cells in the subsequent stage of the culture may be in a range of about 1:1 to about 3:1. Specifically, the ratio of CBMCs to feeder cells may be 2:1.

Another aspect provides a composition for producing a tumor memory killer cell, including IL-2, IL-15, and a feeder cell obtained by irradiating a solid cancer cell or a hematologic cancer cell with radiation.

In an embodiment, the concentration of IL-2 included in the composition may be in a range of about 5 ng/ml to about 50 ng/ml, about 5 ng/ml to about 40 ng/ml, about 5 ng/ml to about 30 ng/ml, about 5 ng/ml to about 20 ng/ml, or about 5 ng/ml to about 10 ng/ml, but the present disclosure is not limited thereto. Specifically, IL-2 may promote the differentiation of CBMCs into tumor memory killer cells.

In an embodiment, the concentration of IL-15 included in the composition may be in a range of about 5 ng/ml to about 50 ng/ml, about 5 ng/ml to about 40 ng/ml, about 5 ng/ml to about 30 ng/ml, about 5 ng/ml to about 20 ng/ml, or about 5 ng/ml to about 10 ng/ml, but the present disclosure is not limited thereto. Specifically, IL-15 may promote the differentiation of CBMCs into tumor memory killer cells.

In an embodiment, the composition may promote the differentiation of CBMCs into tumor memory killer cells. Specifically, produced tumor memory killer cells may increase the proportion of CD3-CD56+ cells compared to cord blood-derived cytokine-induced killer cells, or may increase the phenotype of CD45RO rather than CD45RA. In addition, the tumor memory killer cells induce cancer cell-specific apoptosis through the same mechanism as existing cytokine-induced killer cells, but may exhibit a stronger effect of inducing tumor apoptosis by improving memory ability for tumor cells.

In an embodiment, the composition may include appropriate proteins, cytokines, antibodies, compounds, and other components, provided that they do not impair the effectiveness of producing tumor memory killer cells.

In an embodiment, tumor memory killer cells cultured by the composition may have one or more characteristics selected from (a) to (c) below compared to cytokine-induced killer cells:
(a) an increase in the expression of one or more surface antigen proteins selected from the group consisting of Nkp30, Nkp44, and Nkp46;
(b) an increase in the expression of surface antigen protein CD45RO; and
(c) a decrease in the expression of surface antigen protein CD45RA.

Another aspect provides a composition for treating cancer, including, as an active ingredient, a tumor memory killer cell produced by the method of producing a tumor memory killer cell, or a population thereof.

In an embodiment, the composition may include about 0.5×10⁴ cells/ml to about 2×10⁶ cells/ml. Specifically, the number of cells included per 1 ml may be appropriately adjusted and modified by one of ordinary skill in the art to prevent depletion of nutrients in a culture medium of the cells and achieve maximum expansion and survival.

Another aspect provides a cellular therapeutic agent or pharmaceutical composition including, as an active ingredient, a tumor memory killer cell produced by the method or a population thereof.

The term “cellular therapeutic agent” as used herein refers to a pharmaceutical product used for the purpose of treatment, diagnosis, and prevention, obtained through a series of actions, including growing and screening living autologous, allogenic, or xenogenic cells in vitro or changing the biological characteristics of cells by any other methods to restore the function of cells and tissues. The United States and Korea have been managing cellular therapeutic agents as pharmaceuticals since 1993 and 2002, respectively. These cellular therapeutic agents can be broadly classified as two fields. The first is a stem cell therapeutic agent for tissue regeneration or organ function recovery, and the second is an immune cell therapeutic agent for regulating immune responses, such as suppression of immune responses in vivo or enhancement of immune responses.

In an embodiment, the cellular therapeutic agent or the pharmaceutical composition may be used for the prevention or treatment of cancer.

Another aspect provides use of the tumor memory killer cell or a population thereof for the preparation of a medicine.

Another aspect provides a method of treating a disease, including administering the tumor memory killer cell or a population thereof to a subject.

The term “disease” as used herein may refer to a pathological condition, particularly, cancer, an infectious disease, an inflammatory disease, a metabolic disease, an autoimmune disease, a degenerative disease, a disease related to apoptosis, and graft rejection.

The term “treatment” as used herein may refer to or include alleviation, inhibition of progression, or prevention of a disease, disorder or condition, or one or more symptoms thereof, and the term “active ingredient” or “pharmaceutically effective amount” as used herein may mean any amount of a composition used in the practice of the disclosure provided herein, which is sufficient to alleviate, inhibit the progression of, or prevent a disease, disorder or condition, or one or more symptoms thereof.

The terms “administering,” “introducing,” and “implanting” as used herein are used interchangeably, and may refer to the placement of a composition according to an embodiment into a subject by a method or via a rout that results in at least partial localization of the composition according to an embodiment to a desired site. A composition according to an embodiment may be administered via any suitable route that delivers at least a portion of a cell or a cellular component to a desired location in a living subject. A survival period of the cells after being administered to a subject may be as short as several hours, for example, from about 24 hours to about several days, or as long as several years.

The administration may be administration in combination with an additional anticancer agent or a cytokine agent. Examples of additional anticancer agent may include alkylating agents, antimetabolites, spindle inhibitor plant alkaloids, cell dysfunctional/antitumor antibiotics, topoisomerase inhibitors, antibodies, photosensitizers, and kinase inhibitors. Examples of the anticancer agents may include compounds used in targeted therapy and conventional chemotherapy. In addition, examples of the antibody include alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

The term “produced cell,” for example, “produced tumor memory killer cell” or “isolated activated lymphocyte” as used herein refers to a tissue from which a cell originates, for example, a cell substantially produced or isolated from cord blood.

A composition according to an aspect may be used to treat or prevent a tumor or cancer derived from a neoplasm. Neoplasms may be malignant or benign, and cancers may be primary or metastatic. A neoplasm or cancer may be in an early or late stage. Non-limiting examples of neoplasms or cancers that can be treated may include one or more selected from the group consisting of glioma, gastrointestinal stromal tumor, leukemia, breast cancer, uterine cancer, cervical cancer, gastric cancer, colon cancer, prostate cancer, ovarian cancer, lung cancer, laryngeal cancer, rectal cancer, liver cancer, gallbladder cancer, pancreatic cancer, kidney cancer, skin cancer, bone cancer, muscle cancer, adipose cancer, fibrocyte cancer, hematologic cancer, lymphoma, and multiple myeloma, and may include, for example, one or more selected from the group consisting of glioma, gastrointestinal stromal tumor, leukemia, breast cancer, uterine cancer, cervical cancer, gastric cancer, colon cancer, prostate cancer, and ovarian cancer.

In an embodiment, the method of administering the pharmaceutical composition is not particularly limited, and the pharmaceutical composition may be administered orally or parenterally, such as intravenous, subcutaneous, intraperitoneal, inhalation or topical application, depending on the desired method. The dosage varies depending on the patient's weight, age, gender, health condition, diet, administration time, administration method, excretion rate, severity of the disease, and the like. A daily dosage refers to an amount of a therapeutic substance according to an aspect sufficient to treat a disease condition that is alleviated by administration to a subject in need thereof. An effective amount of a therapeutic agent varies depending on a particular compound, a disease condition and the severity thereof, and a subject in need of treatment, and may be generally determined by one of ordinary skill in the art. As a non-limiting example, the dosage of the composition according to an aspect to the human body may vary depending on the patient's age, weight, gender, dosage form, health condition, and the severity of a disease. The composition may be administered once a day or in multiple doses at regular time intervals, or may be administered multiple times at regular time intervals, at a dose of, for example, about 1,000 cells/time to about 10,000 cells/time, about 1,000 cells/time to about 100,000 cells/time, about 1,000 cells/time to about 1,000,000 cells/time, about 1,000 cells/time to about 10,000,000 cells/time, about 1,000 cells/time to about 100,000,000 cells/time, about 1,000 cells/time to about 1,000,000,000 cells/time, about 1,000 cells/time to about 10,000,000,000 cells/time, or about 1,000 cells/time to about 100,000,000,000, with respect to an adult patient weighing 70 kg.

The term “subject” as used herein refers to a subject in need of treatment for a disease, and more specifically, refers to primates that are humans or non-humans, and mammals such as mice, rats, dogs, cats, horses, and cows.

A pharmaceutical composition according to an embodiment may include a pharmaceutically acceptable carrier and/or an additive. For example, the pharmaceutical composition may include sterile water, physiological saline, common buffers (phosphoric acid, citric acid, other organic acids, and the like), stabilizers, salts, antioxidants (ascorbic acid and the like), surfactants, suspending agents, isotonic agents, preservatives, or the like. For topical administration, the pharmaceutical composition may include a combination with organic substances, such as biopolymers, or inorganic substances, such as hydroxyapatite, specifically collagen matrices, polylactic acid polymers or copolymers, polyethylene glycol polymers or copolymers, and chemical derivatives thereof. When the pharmaceutical composition according to an embodiment is prepared in a formulation suitable for injection, immune cells or substances that increase the activity thereof may be dissolved in a pharmaceutically acceptable carrier or frozen in a dissolved solution state.

The pharmaceutical composition according to an embodiment, if needed according to the administration method or dosage form, may appropriately include a suspending agent, a solubilizing agent, a stabilizer, an isotonic agent, a preservative, an anti-adsorption agent, a surfactant, a diluent, an excipient, a pH adjuster, an analgesic agent, a buffer, a reducing agent, an antioxidant, and the like. Pharmaceutically acceptable carriers and agents suitable for the present disclosure, including those listed above as examples, are described in detail in [Remington's Pharmaceutical Sciences, 19th ed., 1995]. The pharmaceutical composition according to an embodiment may be formulated in unit dosage form by using a pharmaceutically acceptable carrier and/or excipient or prepared by being inserted into a multi-dose container, according to a method that can be easily carried out by one of ordinary skill in the art to which the present disclosure pertains. The dosage form may be in the form of a solution in an oil or aqueous medium, suspension or emulsion, or in the form of powder, a granule, a tablet, or a capsule.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view illustrating a method of producing a tumor memory killer cell, according to an embodiment;

FIG. 2 illustrates graphs showing the results of analysis of CD3 and CD56 phenotypes of tumor memory killer cells compared to cord blood-derived cytokine-induced killer cells, according to an embodiment;

FIG. 3 illustrates graphs showing the results of analysis of Nkp30, Nkp44 and Nkp46 phenotypes of cord blood-derived tumor memory killer cells compared to cord blood-derived cytokine-induced killer cells, according to an embodiment;

FIG. 4 illustrates graphs showing the results of analysis of CD45RA and CD45RO phenotypes of tumor memory killer cells compared to cord blood-derived cytokine-induced killer cells, according to an embodiment;

FIGS. 5A to 5C illustrate the results of transcriptome analysis of cytokine-induced killer (CIK) cells and tumor memory killer cells (TMKCs), in which FIG. 5A is a graph showing the results of confirming that CIK cells and TMKCs are different cells, through the principal component analysis of CIK cells and TMKCs, FIG. 5B illustrates upregulated genes in TMKCs compared to CIK cells, and FIG. 5C is a graph showing the results of gene set enrichment analysis of CIK cells and TMKCs;

FIG. 6 illustrates graphs showing the cytotoxicity of cord blood-derived tumor memory killer cells against solid cancer cells, compared to cord blood-derived cytokine-induced killer cells, according to an embodiment (exNK: expanded natural killer cell), CIK: cytokine-induced killer cell, TMKC: tumor memory killer cell);

FIG. 7 illustrates graphs showing the cytotoxicity of cord blood-derived tumor memory killer cells against hematologic cancer cells, compared to cord blood-derived cytokine-induced killer cells, according to an embodiment (exNK: expanded natural killer cell, CIK: cytokine-induced killer cell, TMKC: tumor memory killer cell); and

FIG. 8 illustrates graphs showing apoptotic effects by using target cells and effector cells (CIK, TMKC) collected from gastric cancer patients.

FIG. 9 illustrates the results of confirming the cytotoxicity of TMKCs produced from the ascites of gastric cancer cells in the peripheral blood of the gastric cancer patients.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, exemplary examples will be presented to aid in understanding of the present disclosure. However, the following examples will be provided to facilitate the understanding of the present disclosure and are not intended to limit the scope of the present disclosure. As embodiments allow for various changes, embodiments are not be limited to the examples disclosed below and may be implemented in various different forms.

### Example 1. Preparation of Activated Lymphocytes

### 1.1. Isolation of Cord Blood Mononuclear Cells (CBMCs)

To isolate activated lymphocytes in cord blood mononuclear cells (CBMCs), preparation started with 80 mL to 100 mL of cord blood collected at the time of delivery. The collected blood was transferred to a 50 mL test tube and diluted with phosphate buffer solution (PBS) containing 2% FBS in a ratio of 1:1, and then 30 mL of the diluted blood was dispensed into a 50 mL test tube with lymphoprep dispensed therein without mixing the layers together, followed by centrifugation at a rotational speed of 1,200×g and at room temperature for 20 minutes without breaks.

The upper plasma layer was slowly removed to prevent the cells of the lymphocyte layer from being sucked in, and the separated monocyte layer was transferred to a 50 mL test tube. The separated monocyte layer was diluted with 50 mL of PBS containing 2% FBS, centrifuged at a rotational speed of 1,500 rpm and at room temperature for 10 minutes, and then the supernatant was removed and the collected cells were dispersed. The cells in the test tube were washed twice with PBS containing 2% FBS. Cells were collected by centrifugation at a rotational speed of 1,500 rpm and at room temperature for 5 minutes, and then the supernatant was removed, and the cells were suspended by using a pipette to disperse the cells well.

### 1.2. Preparation of Feeder Cells

To produce tumor memory killer cells, ascites from patients with solid cancer and a leukapheresis product from patients with hematologic cancer were used as feeder cells to be co-cultured with CBMCs. The patient-derived specimens and cancer cells were irradiated with 50 Gy of radiation to be used as feeder cells.

### Example 2. Culture of Activated Lymphocytes

### 2.1. Culture of Activated Lymphocytes

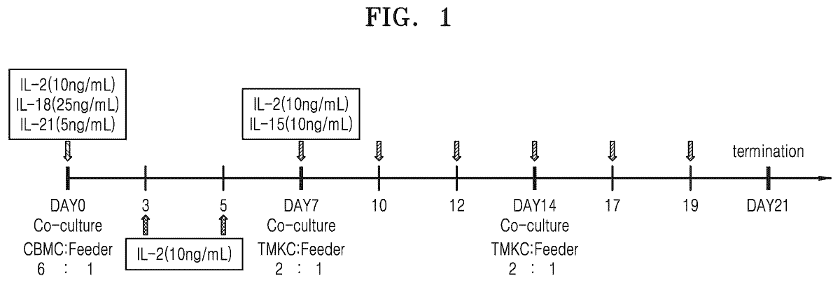

The CBMCs and feeder cells prepared in Example 1. above were used to produce tumor memory killer cells and cytokine-induced killer cells, and a specific production method is as illustrated in FIG. 1.

Specifically, for the production of tumor memory killer cells, 3×10⁶ cells of isolated monocytes on day 0 of culture and 5×10⁵ radiated patient-derived feeder cells were inoculated into a 24-well plate in a ratio of 6:1, and an NK-Xpander™ culture medium containing IL-2 (10 ng/ml), IL-21 (5 ng/ml), and IL-18 (25 ng/ml) was added to culture the cells. Before day 7 of the culture, the culture medium was supplemented with IL-2 (10 ng/ml) every 2 days or 3 days. After day 7 of the culture, lymphocytes and radiated patient-derived feeder cells were re-inoculated into a T-75 or T-175 flask in a ratio of 2:1, and IL-2 (10 ng/ml) and IL-15 (10 ng/mL) were added to co-culture the cells. IL-2 (10 ng/ml) and IL-15 (10 ng/mL) were supplemented every 2 days or 3 days. After day 14 of the culture, lymphocytes and radiated patient-derived feeder cells were re-inoculated into a T-225 flask in a ratio of 2:1, and IL-2 (10 ng/mL) and IL-15 (10 ng/ml) were added to co-culture the cells. Similarly, IL-2 (10 ng/ml) and IL-15 (20 ng/ml) were supplemented every 2 days or 3 days. To prevent nutrient depletion and achieve maximum expansion and survival, the number of cells was maintained at 2×10⁶ cells/mL or less.

Specifically, for the production of cytokine-induced killer cells, 3×10⁶ cells of isolated monocytes on day 0 of culture and an NK-Xpander™ culture medium containing IFN-γ (1000 IU/mL) were added to a 24-well plate to culture the monocytes. On day 1 of the culture, OKT3 (50 ng/ml) and IL-2 (10 ng/ml) were added. Subsequently, the culture medium was supplemented with IL-2 (10 ng/mL) every 2 days or 3 days. To prevent nutrient depletion and achieve maximum expansion and survival, the number of cells was maintained at 2×10⁶ cells/mL or less.

FIG. 1 is a schematic view illustrating a method of producing a tumor memory killer cell, according to an embodiment.

### 2.2. Cell Collection

After day 21 of the culture, the culture was terminated, 250 ml of a cell culture from one T-225 flask was transferred to five 50 mL test tubes. Cells were collected by centrifugation at a rotational speed of 1,500 rpm and at room temperature for 5 minutes, and then the supernatant was removed, and the cells were suspended by using a pipette to disperse the cells well. For the remaining flask, the cell culture was collected in five identical test tubes and centrifuged under the same conditions, and then the supernatant was removed and the cells were well dispersed. The cells in the test tubes were washed twice with PBS. The cells were collected by centrifugation at a rotational speed of 1,500 rpm and at room temperature for 5 minutes, and then the supernatant was removed, and the cells were suspended by using a pipette to disperse the cells well, thereby completing the preparation of activated lymphocytes.

### Experimental Example 1. Cell Phenotypic Analysis of Prepared Activated Lymphocytes

To confirm a degree of distribution of tumor memory killer cells in the activated lymphocytes, some samples of the cultured cells were collected and the cell phenotype was analyzed by fluorescence-activated cell sorting (FACS).

Specifically, IgG1-FITC, IgG1-PE, and IgG1-pe-cy7 were prepared as negative control antibodies, and anti-CD3-FITC, anti-CD8-PE, anti-CD8-FITC, anti-CD56-PE, anti-CD56 Pe-cy7, and anti-NKG2D-PE were prepared as experimental group antibodies. In this case, the same antibodies with different fluorophores were used to prevent overlapping of fluorescence during double staining and triple staining. PBS containing 0.5% albumin was used as the buffer.

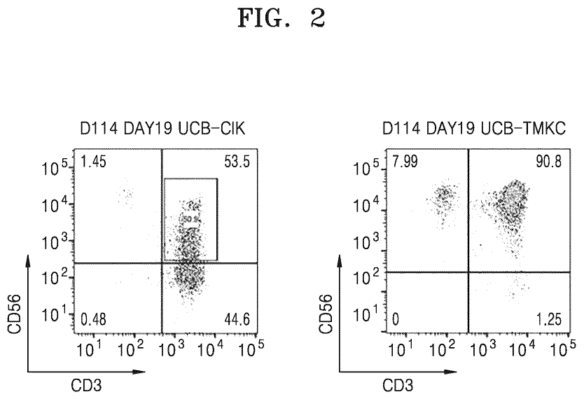

The number of tumor memory killer cells contained in the culture medium was counted, and the tumor memory killer cells were prepared at a concentration of 0.5×10⁷ cells/mL. After centrifugation at a rotational speed of 1,500 rpm and at room temperature for 5 minutes, the supernatant was removed and the buffer was added so that a cell concentration became 0.5×10⁷ cells/mL. 5 mL test tubes were prepared, and then 100 μL of 0.5×10⁷ cells/mL of a cell suspension was added to each tube and the control antibodies and the experimental group antibodies were added. These were mixed well, and then a reaction was allowed to occur therebetween at room temperature for 30 minutes in a dark state, and then 1 mL of buffer was added, followed by centrifugation at a rotational speed of 1,500 rpm and at room temperature for 5 minutes. The supernatant was removed, and then 500 μl of buffer was added and the cell phenotype was analyzed by FACS, and the results thereof are illustrated in FIG. 2.

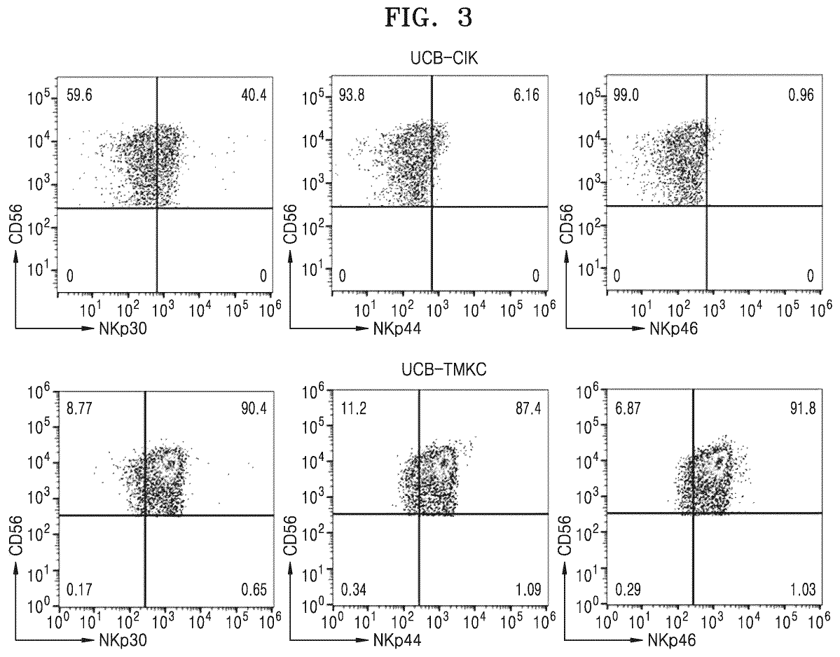

The same method was used to analyze the expression of Nkp 30, Nkp 44, and Nkp 46, which are immune receptors, of cord blood-derived tumor memory killer cells compared to cord blood-derived cytokine-induced killer cells. Specifically, IgG1-FITC, IgG1-PE, and IgG1-pe-cy7 were prepared as negative control antibodies, and anti-CD3-FITC, anti-CD56 Pe-cy7, anti-NKp30-PE, anti-NKp44-PE, and anti-NKp46-PE were prepared as experimental group antibodies. In this case, the same antibodies with different fluorophores were used to prevent overlapping of fluorescence during double staining and triple staining. Similarly, some samples of the cultured cells were collected and the cell phenotype was analyzed by FACS, and the results thereof are illustrated in FIG. 3.

FIG. 2 illustrates graphs showing the results of analysis of CD3 and CD56 phenotypes of cord blood-derived tumor memory killer cells compared to cord blood-derived cytokine-induced killer cells, according to an embodiment.

FIG. 3 illustrates graphs showing the results of analysis of Nkp30, Nkp44 and Nkp46 phenotypes of cord blood-derived tumor memory killer cells compared to cord blood-derived cytokine-induced killer cells, according to an embodiment.

As illustrated in FIG. 2, tumor memory killer cells (90.8%) had the highest proportion of CD56+CD3+ cells, and the proportion of CD56+CD3− cells, which is a reference for measuring the purity of NK cells, was much higher in tumor memory killer cells (7.99%) than that of cord blood-derived cytokine-induced killer cells (1.45%).

As illustrated in FIG. 3, tumor memory killer cells were measured to have significantly higher proportions of Nkp30 (90.4%), Nkp44 (87.4%), and Nkp46-positive (+) (91.8%) compared to cord blood-derived cytokine-induced killer cells.

### Experimental Example 2. Memory Phenotypic Analysis of Prepared Activated Lymphocytes

To confirm the tumor memory-related effector memory phenotype of tumor memory killer cells in the activated lymphocytes, some samples of the cultured cells were collected and the cell phenotype was analyzed by FACS.

Specifically, IgG1-FITC and IgG1-PE were prepared as negative control antibodies, and anti-CD45RA-FITC and anti-CD45RO-PE were prepared as experimental group antibodies. In this case, the same antibodies with different fluorophores were used to prevent overlapping of fluorescence during double staining and triple staining. PBS containing 0.5% albumin was used as the buffer.

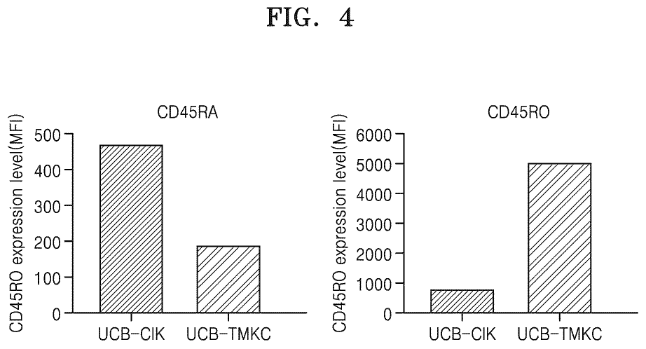

The number of tumor memory killer cells contained in the culture medium was counted, and the tumor memory killer cells were prepared at a concentration of 0.1×10⁶ cells/mL. After centrifugation at a rotational speed of 1,500 rpm and at room temperature for 5 minutes, the supernatant was removed and the buffer was added so that a cell concentration became 0.1×10⁶ cells/mL. 5 mL test tubes were prepared, and then 100 μL of 0.1×10⁶ cells/mL of a cell suspension was added to each tube and the control antibodies and the experimental group antibodies were added. These were mixed well, and then a reaction was allowed to occur therebetween at room temperature for 30 minutes in a dark state, and then 1 mL of buffer was added, followed by centrifugation at a rotational speed of 1,500 rpm and at room temperature for 5 minutes. The supernatant was removed, and then 500 μl of buffer was added and the cell phenotype was analyzed by FACS, and the expression levels of surface markers of the activated lymphocytes were quantified using the mean fluorescence intensity (MFI) value. The results thereof are shown in Table 1 and FIG. 4.

TABLE 1

| | CD45RA (MFI | CD45RO (MFI |
|---|---|---|
| | value) | value) |
| UCB-CIK | 473 | 703 |
| UCK-TMKC | 187 | 4991 |

FIG. 4 illustrates graphs showing the results of analysis of CD45RA and CD45RO phenotypes of tumor memory killer cells compared to cord blood-derived cytokine-induced killer cells, according to an embodiment.

As illustrated in FIG. 4, the expression of CD45RA, which is mainly expressed in naive T cells, was relatively higher in cord blood-derived cytokine-induced killer cells, whereas the expression of CD45RO, which is mainly expressed in effector memory cells, memory T cells, and activated T cells, was measured to be high in tumor memory killer cells.

Through the above results, it was confirmed that tumor memory killer cells have significantly improved tumor memory ability compared to cord blood-derived cytokine-induced killer cells.

### Experimental Example 3. Transcriptome Analysis of Cytokine-Induced Killer (CIK) Cells and Tumor Memory Killer Cells (TMKCs)

The transcriptomes of cytokine-induced killer (CIK) cells and tumor memory killer cells (TMKCs) were analyzed through RNA sequencing.

Specifically, RNA was extracted from each of the CIK cells and TMKCs cultured until day 28 and the transcriptomes thereof were analyzed through RNA sequencing. Specifically, sample QC, DEGs analysis, and functional pathway analysis were performed. Sample QC was performed to confirm good clustering between samples in each of the CIK and TMKC groups through PCA plot and hierarchical clustering. DEG analysis was performed by cutoff using |Fold change|≥2 & padjust<0.01. Over representation analysis (ORA) and gene set enrichment analysis were performed as functional pathway analyses.

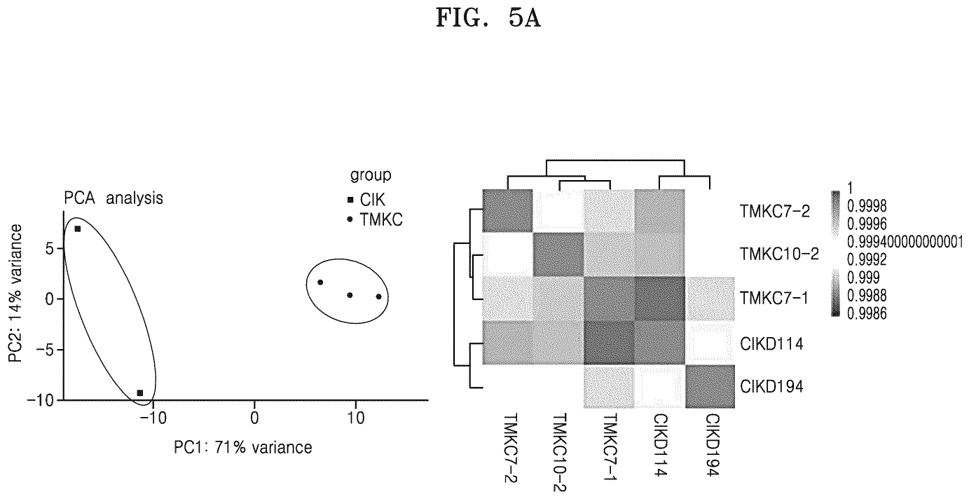

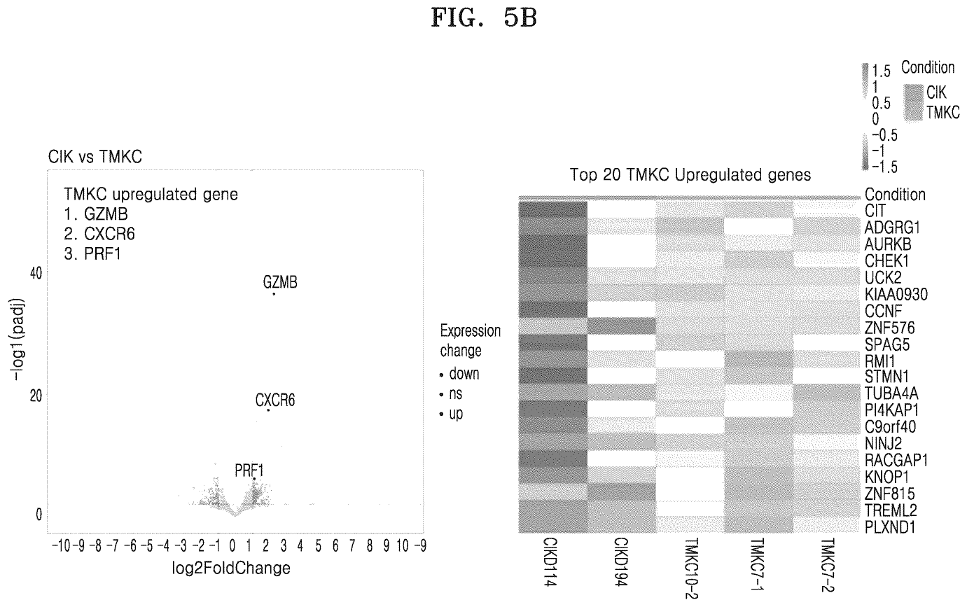

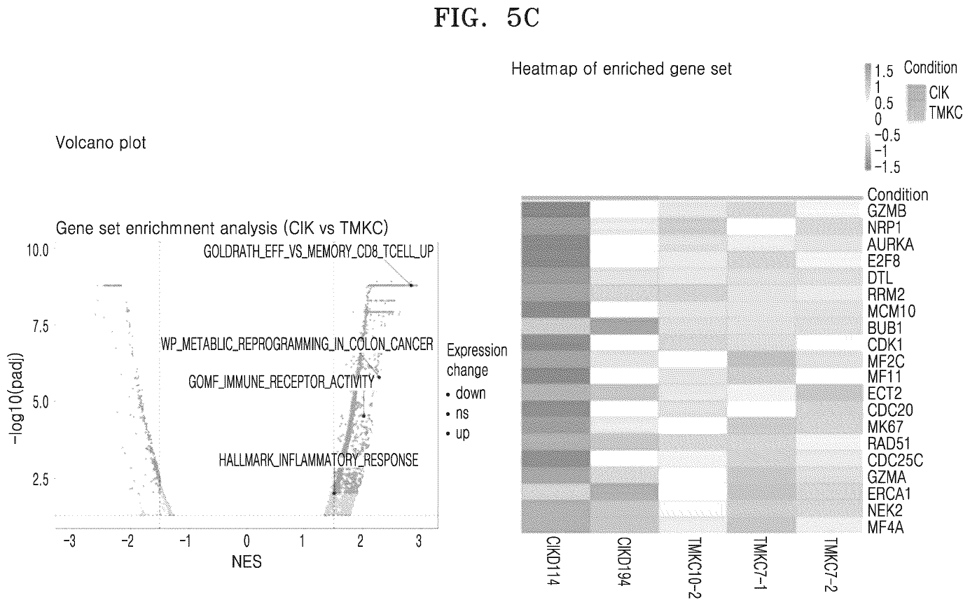

The results thereof are illustrated in FIG. 5.

FIG. 5 illustrates the results of transcriptome analysis of cytokine-induced killer (CIK) cells and tumor memory killer cells (TMKCs), in which FIG. 5A is a graph showing the results of confirming that CIK cells and TMKCs are different cells, through the principal component analysis of CIK cells and TMKCs, FIG. 5B illustrates upregulated genes in TMKCs compared to CIK cells, and FIG. 5C is a graph showing the results of gene set enrichment analysis of CIK cells and TMKCs.

As illustrated in FIG. 5, it was confirmed that CIK cells and TMKCs were different cells, and compared to the CIK cells, the GZMB and PRF1 genes, which are important granules in killing target cells, and the CXCR6 gene, which is important in cell migration, were highly expressed in the TMKCs, and the TMKCs had a memory CD8 T cell-related gene set. It was also confirmed that, as a result of immune signature gene sets, memory T cell upregulated gene sets were enriched in the TMKCs. As a result of GSEA analysis, which performs functional analysis on all genes, it was confirmed that gene sets related to TCR/γδ T cells were enriched in the TMKC group.

### Experimental Example 4. Cytotoxicity Analysis of Prepared Activated Lymphocytes Against Solid Cancer Cells

To confirm the effectiveness of tumor memory killer cells in the activated lymphocytes, as an anticancer cellular therapeutic agent, the degree of killing cancer cells when ascites cells from solid cancer patients were treated therewith was analyzed by FACS.

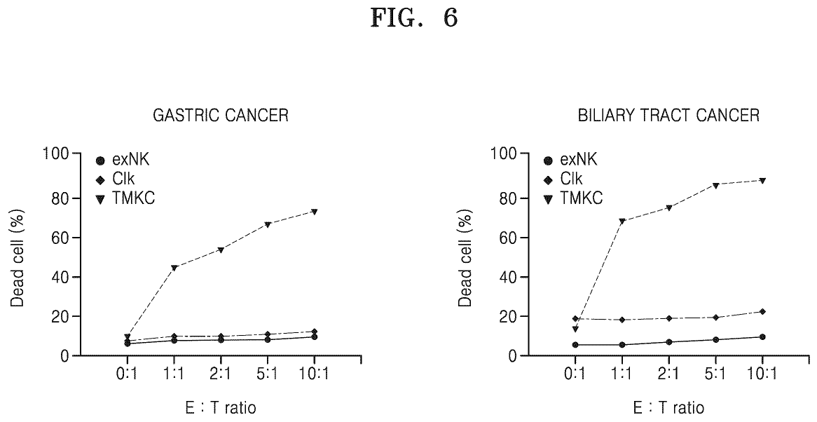

Specifically, ascites from patients with solid cancer (gastric cancer and biliary tract cancer) as target (T) cells was centrifuged at 1500 rpm for 5 minutes, and the supernatant was removed, followed by washing with DPBS. The washed cells were prepared in NKxpander™ culture medium at a concentration of 1×10⁵ cells/ml. The prepared cells were treated with CFSE (#c34554, Invitrogen) and incubated in a CO₂ incubator for 30 minutes. Subsequently, the resulting cells were washed twice with DPBS, and then co-cultured with tumor memory killer cells (TMKCs) produced according to the method of the present disclosure as effector (E) cells or expanded natural killer (exNK) cells or cytokine-induced killer (CIK) cells as a control in a ratio of effector cells to target cells (E:T) of 10:1, 5:1, 2:1, or 1:1, followed by incubation in a CO₂ incubator for 4 hours. After incubation was terminated, the cells were treated with FVD (#L34973, Invitrogen) and incubated in a 4° C. refrigerator for 30 minutes. After incubation was completed, the cells were washed twice with DPBS. The cells were collected in test tubes and the ability thereof to kill cancer cells was analyzed by FACS. The results thereof are illustrated in FIG. 6.

FIG. 6 illustrates graphs showing the cytotoxicity of tumor memory killer cells against solid cancer cells, compared to cord blood-derived cytokine-induced killer cells, according to an embodiment (exNK: expanded natural killer cell), CIK: cytokine-induced killer cell, TMKC: tumor memory killer cell).

As illustrated in FIG. 6, high cytotoxicity selectively against solid cancer was confirmed in tumor memory killer cells compared to cord blood-derived cytokine-induced killer cells.

### Experimental Example 5. Cytotoxicity Analysis of Prepared Activated Lymphocytes Against Acute Myeloid Leukemia Cells

To confirm the effectiveness of tumor memory killer cells in the activated lymphocytes, as an anticancer cellular therapeutic agent, the degree of killing cancer cells when blood cells from patients with acute myeloid leukemia were treated therewith was analyzed by FACS.

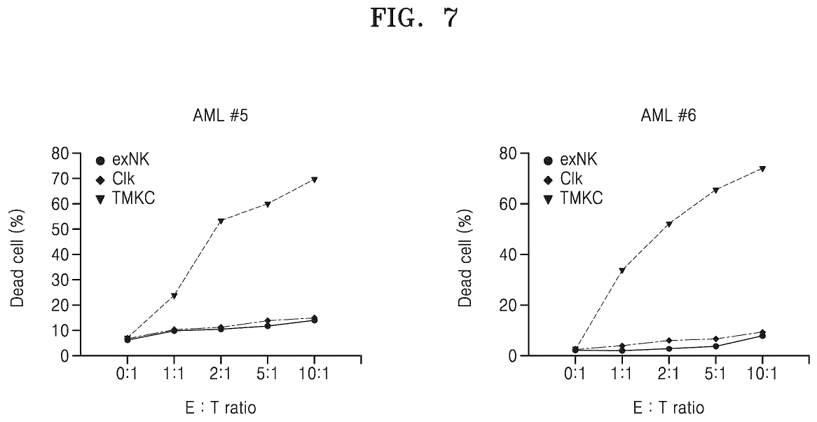

Specifically, blood cells from patients with acute myeloid leukemia as target (T) cells were centrifuged at 1500 rpm for 5 minutes, and the supernatant was removed, followed by washing with DPBS. The washed cells were prepared in NKxpander™ culture medium at a concentration of 1×10⁵ cells/ml. The prepared cells were treated with CFSE (#34554, Invitrogen) and incubated in a CO₂ incubator for 30 minutes. Subsequently, the resulting cells were washed twice with DPBS, and then co-cultured with tumor memory killer cells (TMKCs) produced according to the method of the present disclosure as effector (E) cells or expanded natural killer (exNK) cells or cytokine-induced killer (CIK) cells as a control in a ratio of effector cells to target cells (E:T) of 10:1, 5:1, 2:1, or 1:1, followed by incubation in a CO₂ incubator for 4 hours. After the incubation, the cells were treated with FVD (#L34973, Invitrogen) and incubated in a 4° C. refrigerator for 30 minutes. After incubation was completed, the cells were washed twice with DPBS. The cells were collected in test tubes and the ability thereof to kill cancer cells was analyzed by FACS. The results thereof are illustrated in FIG. 7.

FIG. 7 illustrates graphs showing the cytotoxicity of tumor memory killer cells against hematologic cancer cells, compared to cord blood-derived cytokine-induced killer cells, according to an embodiment (exNK: expanded natural killer cell), CIK: cytokine-induced killer cell, TMKC: tumor memory killer cell).

As illustrated in FIG. 7, high cytotoxicity selectively against acute myeloid leukemia was confirmed in tumor memory killer cells compared to cord blood-derived cytokine-induced killer cells.

Through the above results, tumor memory killer cells induce apoptosis through the same mechanism as cord blood-derived cytokine-induced killer cells, but exhibit a strong effect of inducing apoptosis in solid cancer and hematologic cancer through significantly improved tumor memory ability, and thus can be effectively used as an anticancer immune cell therapeutic agent.

### Experimental Example 6. Confirmation of Selective Cytotoxicity of Produced TMKCs Against Gastric Cancer Cells

### 6.1 Collection of Samples (Target Cells) and Confirmation of Selective Cytotoxicity of TMKCs Against Allo PBMCs

10 ml of blood from gastric cancer patients was collected, followed by lymphoprep, and then only MNCs were isolated.

Selective cytotoxicity of TMKCs against gastric cancer cells was examined using the TMKCs produced by the method of Example 2 and target cells collected by the method of Example 6.1.

Specifically, to perform cytotoxicity analysis, PBMCs derived from gastric cancer patients, obtained from three different donors, were used as target cells. All target cells were labeled with carboxyfluorescein succinimidyl ester (CFSE), and the effector cells and the target cells were labeled with fixable viability dyes (FVD) and co-cultured for 4 hours to separate dead cells. Dead target cells were quantitatively analyzed in the CFSE+ FVD+ region by using FACS equipment.

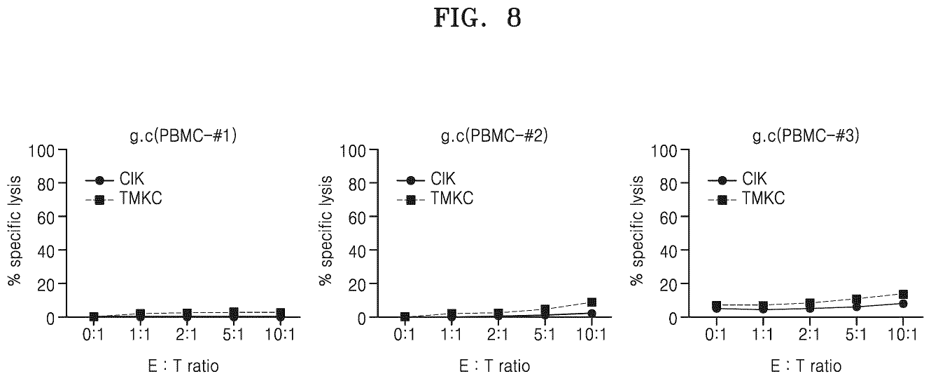

The results thereof are illustrated in FIG. 8.

FIG. 8 illustrates graphs showing apoptotic effects by using target cells collected from gastric cancer patients and effector cells (CIK, TMKC) (X axis: T ratio, Y axis: dead cell percentage).

As illustrated in FIG. 8, it was confirmed that the produced TMKCs selectively killed only cells present in the ascites from gastric cancer patients, but did not kill immune cells present in the peripheral blood (PB) of gastric cancer patients (randomly selected patients).

### 6.2 Confirmation of Selective Cytotoxicity of TMKCs Targeting Native PBMCs

The selective cytotoxicity of TMKCs against gastric cancer cells was examined by using TMKCs produced from the ascites of gastric cancer patients by the method of Example 2 and target cells (peripheral blood of gastric cancer patients) collected using the method of Example 6.1. Specifically, to perform cytotoxicity analysis, ascites and autologous PBMCs from gastric cancer patients were used as target cells. All target cells were labeled with carboxyfluorescein succinimidyl ester (CFSE), and the effector cells and the target cells were labeled with fixable viability dyes (FVD) and co-cultured for 4 hours to separate dead cells. Dead target cells were quantitatively analyzed in the CFSE+ FVD+ region by using FACS equipment.

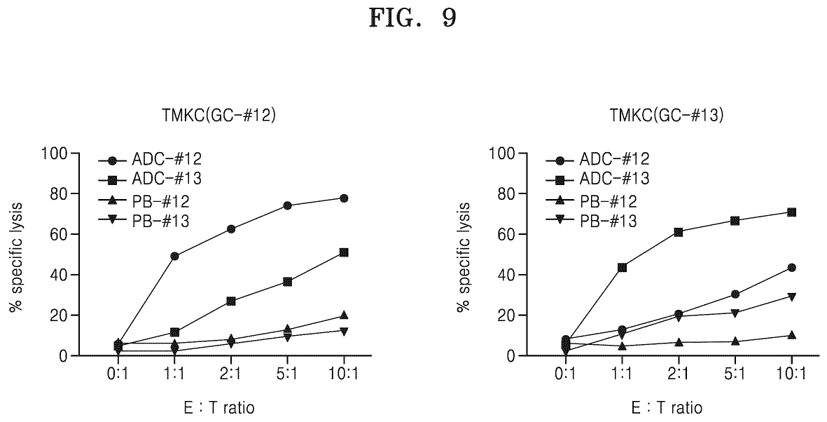

The results thereof are illustrated in FIG. 9.

FIG. 9 illustrates the results of confirming the cytotoxicity of TMKCs produced from the ascites of gastric cancer cells in the peripheral blood of the gastric cancer patients.

As illustrated in FIG. 9, it was confirmed that the TMKCs produced from the ascites of gastric cancer patients did not have high cytotoxicity against immune cells present in the peripheral blood (PB) of the gastric cancer patients.

The foregoing description of the present disclosure is provided for illustrative purposes only, and it will be understood by those of ordinary skill in the art to which the present disclosure pertains that the present disclosure may be easily modified in other particular forms without changing the technical spirit or essential characteristics of the present disclosure. Thus, the above-described examples and experimental examples should be construed as being provided for illustrative purposes only and not for purposes of limitation.

According to a tumor memory killer cell according to an aspect and a method of producing the same, tumor memory killer cells with improved memory ability for tumor cells are provided, and thus can be effectively used as an anticancer immune cell therapeutic agent which induces cancer cell death through the same mechanism as existing cytokine-induced killer cells, but has a stronger effect of inducing tumor cell death.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of producing a tumor memory killer cell, the method comprising:
(a) obtaining a cord blood mononuclear cell (CBMC); and
(b) co-culturing the obtained CBMC and a feeder cell in a medium containing a cytokine.

2. The method of claim 1, wherein the cytokine is one or more selected from the group consisting of IL-2, IL-21, IL-18, and IL-15.

3. The method of claim 1, wherein the cytokine comprises IL-2 at a concentration of about 5 ng/ml to about 30 ng/ml, or IL-15 at a concentration of about 5 ng/ml to about 30 ng/ml.

4. The method of claim 1, wherein the feeder cell is a solid cancer cell or a hematologic cancer cell.

5. The method of claim 4, wherein the solid cancer cell is a cell of colon cancer, ovarian cancer, liver cancer, biliary tract cancer, lung cancer, pancreatic cancer, or prostate cancer.

6. The method of claim 1, wherein the feeder cell is irradiated with about 20 Gy to about 200 Gy of radiation.

7. The method of claim 1, wherein a ratio of the CBMC to the feeder cell in the co-culture is in a range of about 1:1 to about 8:1.

8. The method of claim 1, wherein the co-culture comprises an initial stage for about 2 days to about 7 days and a subsequent stage for about 10 days to about 20 days, wherein a composition of the cytokine contained in the medium in the initial stage is different from a composition of the cytokine contained in the medium in the subsequent stage.

9. The method of claim 8, wherein the cytokine in the initial stage of the co-culture comprises two or more cytokines selected from the group consisting of IL-2, IL-18, and IL-21.

10. The method of claim 8, wherein a ratio of the CBMC to the feeder cell in the initial stage of the co-culture is in a range of about 5:1 to about 8:1.

11. The method of claim 8, wherein the cytokine in the subsequent stage of the co-culture comprises IL-2 and IL-15.

12. The method of claim 8, wherein a ratio of the CBMC to the feeder cell in the subsequent stage of the co-culture is in a range of about 1:1 to about 3:1.

13. The method of claim 1, the tumor memory killer cell that has a surface antigen characteristic of CD56+CD3+, CD45RA−, or CD45RO+.

14. The method of claim 1, the tumor memory killer cell is positive for surface marker Nkp30, Nkp44 or Nkp46.

15. The method of claim 1, the tumor memory killer cell is derived from any one selected from the group consisting of umbilical cord, cord blood, placenta, amniotic fluid, and amniotic membrane.

16. The method of claim 1, wherein an occupancy rate of memory T cells in a group of the isolated tumor memory killer cell is 50% or more.

17. The method of claim 16, wherein an occupancy rate of central memory T cells in the memory T cells is 50% or more.

18. The method of claim 1, wherein the tumor memory killer cell or a population thereof has increased anticancer activity compared to cytokine-induced killer cells, or is genetically modified to have increased anticancer activity, wherein the genetically modified tumor memory killer cell or a population thereof is engineered to express a chimeric antigen receptor (CAR) or a homing receptor.

19. A method of preventing or treating cancer, the method comprising administering, to a subject, a tumor memory killer cell produced by the method according to claim 1 or a population thereof.

20. The composition of claim 19, wherein the cancer is selected from the group consisting of glioma, gastrointestinal stromal tumor, leukemia, breast cancer, uterine cancer, cervical cancer, gastric cancer, colon cancer, prostate cancer, ovarian cancer, lung cancer, laryngeal cancer, rectal cancer, liver cancer, gallbladder cancer, pancreatic cancer, kidney cancer, skin cancer, bone cancer, muscle cancer, adipose cancer, fibrocyte cancer, hematologic cancer, lymphoma, and multiple myeloma.
